(19) United States
(12) Patent Application Publication
(10) Pub. No.: US 2012/0310095 A1
(43) Pub. Date: Dec. 6, 2012

(54) ULTRASOUND DIAGNOSTIC APPARATUS AND ULTRASOUND IMAGE PRODUCING METHOD

(76) Inventor: Tomoo SATO, Kanagawa (JP)

(21) Appl. No.: 13/466,424

(22) Filed: May 8, 2012

(30) Foreign Application Priority Data
Jun. 6, 2011 (JP) 2011-126190

(51) Int. Cl. A61B 8/14

(52) U.S. Cl. 600/447

(57) ABSTRACT

An ultrasound diagnostic apparatus includes reception signal processors having an A/D conversion executable range narrower than an amplitude range of reception signals obtained from an entire depth of a measurement region; and a controller for dividing the measurement region into a plurality of measurement depth zones each allowing reception signals within an amplitude range narrower than the A/D conversion executable range of the reception signal processors, controlling a transmission driver so that transmission of an ultrasonic beam from the array transducer may be performed plural times corresponding to the measurement depth zones, and controlling the reception signal processors so that reception data associated with each measurement depth zone may be acquired based on the ultrasonic beam as transmitted corresponding to the relevant measurement depth zone.

### BACKGROUND OF THE INVENTION

The present invention relates to an ultrasound diagnostic apparatus and an ultrasound image producing method, and particularly to an ultrasound diagnostic apparatus producing an ultrasound image based on reception data acquired by processing in reception signal processors reception signals outputted from an array transducer having received an ultrasonic echo produced by a subject.

In the medical field, ultrasound diagnostic apparatus employing ultrasound images have already been put to practical use. A typical ultrasound diagnostic apparatus for medical use transmits an ultrasonic beam from an array transducer of an ultrasound probe toward the inside of a subject, receives an ultrasonic echo from the subject on the array transducer, and electrically processes a reception signal corresponding to the received echo in an apparatus body so as to generate an ultrasound image.

As an example, JP 4-232888 A discloses an ultrasonic diagnostic system performing reception focusing, on which reception signals outputted from an array transducer having received ultrasonic echo components are each amplified by a preamplifier and subjected to A/D (analog/digital) conversion by an A/D converter to obtain digital reception data, and the obtained digital data are matched to one another in phase by imparting adequate delays to them, and as such added to one another so as to carry out the reception focusing.

According to the reception focusing process as disclosed, a sound ray signal is obtained as a well-focused ultrasonic echo, and the B-mode image signal which is tomographic image information on the inside of a subject is generated based on a plurality of sound ray signals obtained in a region under diagnosis.

### SUMMARY OF THE INVENTION

In such ultrasonography as conducted on the above system, an ultrasonic beam is attenuated while traveling within the subject, so that the ultrasonic beam which reaches a deeper site in the subject will have a lower intensity. In addition, an ultrasonic echo reflected by any site in the subject toward the ultrasound probe is attenuated while traveling within the subject. As a consequence, reception signals outputted from the array transducer will vary in amplitude with measurement depth.

The A/D converter is thus demanded to have a large dynamic range so as to allow reception signals throughout a measurement region, ranging from a reception signal with a larger amplitude corresponding to a shallower zone in the measurement region to a reception signal with a smaller amplitude corresponding to a deeper zone, to be A/D converted with a good resolution. In the conventional ultrasound diagnostic apparatus, however, an A/D converter used is inadequate in dynamic range, which is generally compensated for by combining the preamplifier with a variable gain amplifier, that is to say, by the change of a static gain of the preamplifier and the variation in dynamic gain of the variable gain amplifier.

If, however, the gain of the preamplifier is made too high, an excessive reception signal may be inputted to the A/D converter or recovery from the saturation immediately following the transmission of a reception signal may be reduced with respect to a zone at a shallower location. In addition, the variable gain amplifier does not necessarily bring about a favorable noise figure (NF) because it merely attenuates the amplitude of a reception signal with an attenuator to make it compatible with the dynamic range of the A/D converter. Consequently, reception data acquired from the reception signal as subjected to A/D conversion by the A/D converter may be reduced in precision, leading to the quality reduction of an ultrasound image.

There lies another problem in that, if the gain of the preamplifier is made higher, the bias for class A amplification needs to be increased in order to maintain the linearity, which results in a higher power consumption.

An object of the present invention is to provide an ultrasound diagnostic apparatus and an ultrasound image producing method that resolve such problems of the past and enable producing an ultrasound image of high quality and reduced power consumption while compensating for an inadequate dynamic range of A/D converters.

An ultrasound diagnostic apparatus according to the present invention comprises:

an array transducer;

a transmission driver for transmitting an ultrasonic beam from the array transducer toward a subject;

reception signal processors for acquiring reception data by processing reception signals outputted from the array transducer having received an ultrasonic echo from the subject, the reception signal processors having an A/D conversion executable range narrower than an amplitude range of reception signals obtained from an entire depth of a measurement region;

an image producer for producing an ultrasound image based on the reception data acquired in the reception signal processors; and

a controller for dividing the measurement region into a plurality of measurement depth zones each allowing reception signals within an amplitude range narrower than the A/D conversion executable range of the reception signal processors, controlling the transmission driver so that transmission of an ultrasonic beam from the array transducer may be performed plural times corresponding to the measurement depth zones, and controlling the reception signal processors so that reception data associated with each measurement depth zone may be acquired based on the ultrasonic beam as transmitted corresponding to the relevant measurement depth zone.

An ultrasound image producing method according to the present invention comprises the steps of:

transmitting an ultrasonic beam from an array transducer toward a subject based on a driving signal fed from a transmission driver;

acquiring reception data by processing reception signals outputted from the array transducer in response to ultrasonic echos from an entire depth of a measurement region in reception signal processors having an A/D conversion executable range narrower than an amplitude range of the reception signals;

producing an ultrasound image based on the acquired reception data;

dividing the measurement region into a plurality of measurement depth zones each allowing reception signals within an amplitude range narrower than the A/D conversion executable range of the reception signal processors;

controlling the transmission driver so that transmission of an ultrasonic beam from the array transducer may be performed plural times corresponding to the measurement depth zones; and

controlling the reception signal processors so that reception data associated with each measurement depth zone may be acquired based on the ultrasonic beam as transmitted corresponding to the relevant measurement depth zone.

### BRIEF DESCRIPTION OF THE DRAWINGS

FIG. 1 is a block diagram showing a configuration of an ultrasound diagnostic apparatus according to Embodiment 1 of the present invention;

FIG. 2 is a block diagram showing an internal structure of a reception signal processor used in Embodiment 1;

FIG. 3 is a graph illustrating a relationship between an A/D conversion executable range by the reception signal processor and ultrasonic echos;

FIG. 4 is a graph illustrating the reception of ultrasonic echos in first and second ultrasonic beam transmission and reception processes in Embodiment 1;

FIG. 5 is a graph illustrating the reception of ultrasonic echos in first and second ultrasonic beam transmission and reception processes in Embodiment 2; and

FIG. 6 is a graph illustrating the reception of ultrasonic echos in first and second ultrasonic beam transmission and reception processes in Embodiment 3.

### DETAILED DESCRIPTION OF THE INVENTION

In the following, embodiments of the present invention are described in reference to the accompanying drawings.

### Embodiment 1

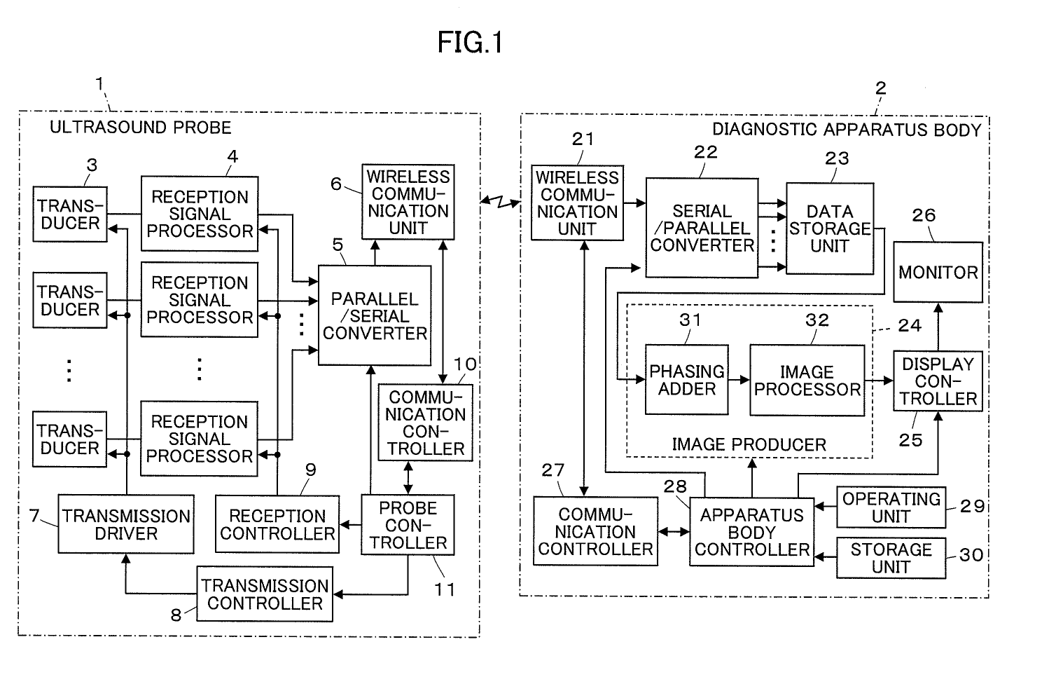

FIG. 1 shows the configuration of an ultrasound diagnostic apparatus according to Embodiment 1. The ultrasound diagnostic apparatus as shown includes an ultrasound probe 1 and a diagnostic apparatus body 2 connected with the ultrasound probe 1 through wireless communication.

The ultrasound probe 1 has a plurality of ultrasound transducers 3 unidimensionally or two-dimensionally arrayed, and connected to corresponding reception signal processors 4, respectively. The reception signal processors 4 are connected to a wireless communication unit 6 via a parallel/serial converter 5. The ultrasound transducers 3 are connected with a transmission controller 8 via a transmission driver 7, while the reception signal processors 4 are connected with a reception controller 9. The wireless communication unit 6 is connected with a communication controller 10. The parallel/serial converter 5, the transmission controller 8, the reception controller 9, and the communication controller 10 are connected with a probe controller 11.

Each of the ultrasound transducers 3 transmits ultrasound in accordance with a driving signal fed from the transmission driver 7, and receives an ultrasonic echo from a subject so as to output a reception signal. Each ultrasound transducer 3 is comprised of a vibrating element having a piezoelectic body and electrodes formed at both ends of the piezoelectric body, with examples of the material for the body including a piezoelectic ceramic typified by lead zirconate titanate (PZT), a polymeric piezoelectric material typified by polyvinylidene fluoride (PVDF), and a piezoelectric single crystal typified by lead magnesium niobate-lead titanate solid solution (PMN-PT).

If a pulsed voltage or a continuous wave voltage is applied across the electrodes of the vibrating element as above, the piezoelectic body expands and contracts, and ultrasound in pulse or continuous wave form is generated from the vibrating element. Ultrasounds generated from the individual vibrating elements are synthesized into an ultrasonic beam. In addition, each vibrating element expands and contracts during the reception of propagating ultrasound to generate an electric signal, and the electric signal is outputted as a reception signal representing the reception of ultrasound.

The transmission driver 7 includes, for instance, a plurality of pulse generators, and is adapted to modify, based on the transmission delay pattern as selected by the transmission controller 8, the delay amounts of the driving signals to be fed to the ultrasound transducers 3 so that ultrasounds transmitted from the ultrasound transducers 3 may form so wide an ultrasonic beam as to cover a specified tissue area in a subject, and then feed the driving signals to the transducers 3.

Each reception signal processor 4 generates a complex baseband signal by subjecting a reception signal outputted from the corresponding ultrasound transducer 3 to quadrature detection or quadrature sampling under the control of the reception controller 9, then performs sampling of the complex baseband signal to generate sample data including information on the tissue area, and feeds the sample data to the parallel/serial converter 5. The reception signal processors 4 may generate sample data by subjecting the data as acquired by the sampling of the complex baseband signal to data compression for low bit rate coding.

The parallel/serial converter 5 converts the sample data in parallel form as generated by the reception signal processors 4 into serial sample data.

The wireless communication unit 6 transmits the serial sample data by modulating a carrier based on the serial sample data to generate a transmission signal, and feeding the transmission signal to an antenna to thereby transmit radio waves from the antenna. Usable modulation methods include amplitude shift keying (ASK), phase shift keying (PSK), quadrature phase shift keying (QPSK), and sixteen quadrature amplitude modulation (16QAM).

The wireless communication unit 6 communicates with the diagnostic apparatus body 2 in a wireless manner so as not only to transmit sample data to the diagnostic apparatus body 2, but receive various control signals from the diagnostic apparatus body 2 to output the received control signals to the communication controller 10. The communication controller 10 controls the wireless communication unit 6 so that transmission of sample data may be performed at the radio field intensity for transmission as specified by the probe controller 11, and outputs the control signals as received by the wireless communication unit 6 to the probe controller 11.

The probe controller 11 controls individual components of the ultrasound probe 1 based on various control signals transmitted from the diagnostic apparatus body 2.

The ultrasound probe 1 has a battery included therein (not shown), from which power is fed to individual circuits in the ultrasound probe 1.

The ultrasound probe 1 may be an external probe, such as of a linear scanning type, of a convex scanning type, and of a sector scanning type, or a probe for endoscopic ultrasonography, such as of a radial scanning type.

The diagnostic apparatus body 2 has a wireless communication unit 21 connected to a data storage unit 23 via a serial/parallel converter 22, with the data storage unit 23 being connected to an image producer 24. The image producer 24 is connected to a monitor 26 via a display controller 25. The wireless communication unit 21 is also connected with a communication controller 27, and the serial/parallel converter 22, the image producer 24, the display controller 25 and the communication controller 27 are connected with an apparatus body controller 28. The apparatus body controller 28 is in turn connected with an operating unit 29 used by an operator to perform input operations, and a storage unit 30 for storing operational programs.

The wireless communication unit 21 communicates with the ultrasound probe 1 in a wireless manner so as to transmit various control signals to the ultrasound probe 1. In addition, the wireless communication unit 21 outputs serial sample data by demodulating a signal received by an antenna.

The communication controller 27 controls the wireless communication unit 21 so that transmission of various control signals may be performed at the radio field intensity for transmission as specified by the apparatus body controller 28.

The serial/parallel converter 22 converts serial sample data outputted from the wireless communication unit 21 into parallel sample data. The data storage unit 23 is comprised of a memory, a hard disk or the like, and adapted to store the sample data as converted by the serial/parallel converter 22 for at least one frame.

The image producer 24 subjects the sample data as read frame by frame from the data storage unit 23 to reception focusing so as to produce an image signal representing an ultrasound diagnostic image. The image producer 24 includes a phasing adder 31 and an image processor 32.

The phasing adder 31 selects, in accordance with the reception direction as specified in the apparatus body controller 28, one reception delay pattern from among those stored in advance, and provides complex baseband signals represented by the sample data with their respective delays based on the selected reception delay pattern, then adds the delayed signals to thereby perform the reception focusing. The reception focusing allows a baseband signal (sound ray signal) as a well-focused ultrasonic echo.

The image processor 32 generates the B-mode image signal which is tomographic image information on a tissue in the subject based on a sound ray signal generated by the phasing adder 31. The image processor 32 includes a sensitivity time control (STC) device and a digital scan converter (DSC). The STC device corrects the sound ray signal for attenuation due to distance in accordance with the depth of the position where ultrasound was reflected. The DSC subjects the sound ray signal as corrected by the STC device to the conversion (raster conversion) into an image signal compatible with the conventional television signal scanning method, and to such image processing as grayscaling as required, so as to generate a B-mode image signal.

The display controller 25 controls the monitor 26 based on an image signal generated by the image producer 24 to display an ultrasound diagnostic image. The monitor 26 includes a display device such as an LCD, and is adapted to display an ultrasound diagnostic image under the control of the display controller 25.

The apparatus body controller 28 controls individual components of the diagnostic apparatus body 2 based on various instruction signals and the like inputted by an operator from the operating unit 29.

In the diagnostic apparatus body 2 as above, the serial/parallel converter 22, the image producer 24, the display controller 25, the communication controller 27, and the apparatus body controller 28 are implemented by a CPU associated with operational programs for giving the CPU instructions on various kinds of processing, while the above components may also be implemented by a digital circuitry. The operational programs are stored in the storage unit 30. As the storage unit 30, a recording medium such as a hard disk, a flexible disk, an MO, an MT, a RAM, a CD-ROM, a DVD-ROM, an SD card, a CF card, or a USB memory, a server, or the like may be used.

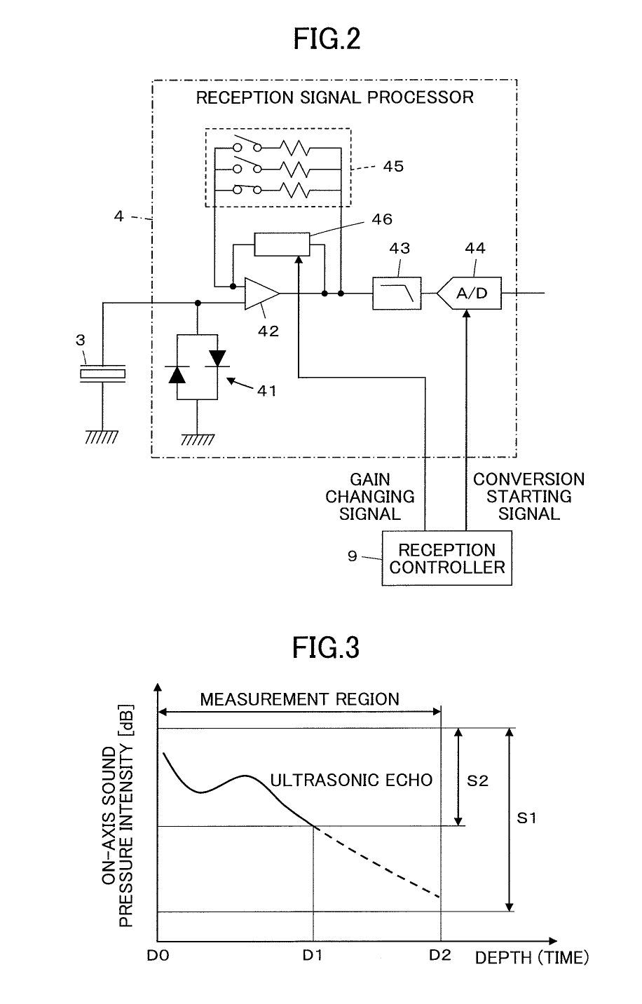

FIG. 2 shows the internal structure of each reception signal processor 4 in the ultrasound probe 1. Each reception signal processor 4 has a preamplifier 42 connected with the corresponding ultrasound transducer 3 via a clip circuit 41 for input protection, and an A/D converter 44 connected with an output terminal of the preamplifier 42 via a low pass filter 43. A gain setting circuit 45 and a gain changing circuit 46 are each connected with the preamplifier 42 in a parallel manner.

The clip circuit 41 prevents a signal at a voltage exceeding a predetermined value from being inputted from the ultrasound transducer 3 to the preamplifier 42. The preamplifier 42 amplifies a reception signal outputted from the ultrasound transducer 3. The gain setting circuit 45 is adapted to set the gain of the preamplifier 42 as appropriate to a subject and its site under diagnosis, and so forth.

The gain changing circuit 46 changes the gain of the preamplifier 42 as set by the gain setting circuit 45, based on a gain changing signal inputted from the reception controller 9.

The low pass filter 43 removes the high frequency components which are not used for signal detection from the reception signal as amplified by the preamplifier 42. The A/D converter 44 converts the reception signal in analog form from which the high frequency components have been removed by the low pass filter 43 into a digital signal based on a conversion starting signal inputted from the reception controller 9.

In the reception signal processor 4 with the structure as above, an A/D conversion executable range for A/D conversion of the reception signal by the reception signal processor 4 is determined, and A/D conversion of a reception signal is performed with a resolution depending on the dynamic range of the A/D converter 44.

As an example, it is assumed that an ultrasonic beam transmission and reception process is to be performed on a measurement region extending from depth DO to depth D2 as shown in FIG. 3. If the A/D conversion executable range by the reception signal processor 4 is set corresponding to so wide an intensity range S1 as to cover the intensities of all the ultrasonic echos returning from the entire depth of the measurement region, reception signals outputted from the ultrasound transducers 3 as a result of the reception of the ultrasonic echos are able to be A/D converted at a time by the A/D converter 44 of the reception signal processor 4. In that case, however, the resolution of A/D conversion is reduced as the A/D conversion executable range is specified corresponding to a wider intensity range if the A/D converter 44 of the reception signal processor 4 has a fixed dynamic range.

If the A/D conversion executable range by the reception signal processor 4 is specified corresponding to an intensity range S2 covering only part of the intensities of ultrasonic echos returning from different sites in the measurement region, A/D conversion is not possible to perform on reception signals generated with respect to the ultrasonic echos as represented with a broken line between depths D1 and D2 that have intensities outside the range corresponding to the A/D conversion executable range, while possible to perform on reception signals generated with respect to the ultrasonic echos as represented with a solid line between depths D0 and D1 with a higher resolution as compared with the case where the A/D conversion executable range is set corresponding to the wide intensity range S1.

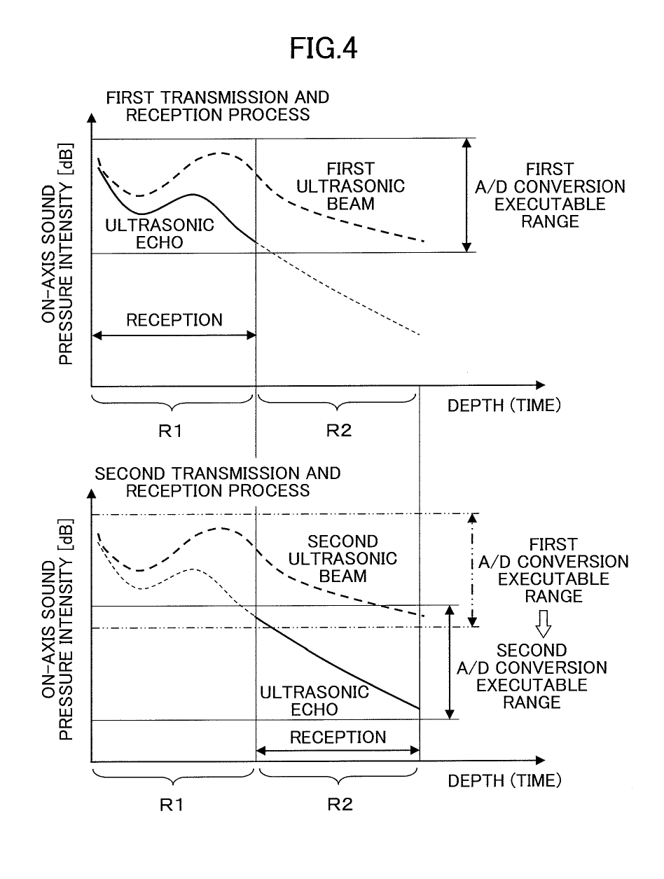

In Embodiment 1, as shown in FIG. 4, the A/D conversion executable range by the reception signal processor 4 is set corresponding to about half an intensity range covering the intensities of all the ultrasonic echos returning from the entire depth of a measurement region, and the measurement region is divided into first and second measurement depth zones R1 and R2, each allowing reception signals within an amplitude range narrower than the A/D conversion executable range by the reception signal processor 4. Then, since all the reception signals generated with respect to the ultrasonic echos resulting from one ultrasonic beam transmission and reception process are not able to be A/D converted at a time, the identical ultrasonic beam is transmitted twice to the first and second measurement depth zones R1 and R2, and ultrasonic echos from the first measurement depth zone R1 and from the second measurement depth zone R2 are received by displacing the A/D conversion executable range by the reception signal processor 4, so as to achieve A/D conversion of all the reception signals associated with the produced ultrasonic echos.

In other words: In a first ultrasonic beam transmission and reception process, a first A/D conversion executable range corresponding to about half an intensity range covering the intensities of all the ultrasonic echos from the entire depth of the measurement region, and also corresponding to the intensity range of ultrasonic echos from the first measurement depth zone R1 is selected. On the other hand, in a second ultrasonic beam transmission and reception process, a second A/D conversion executable range having the same bit width as the first A/D conversion executable range and, at the same time, corresponding to the intensity range of ultrasonic echos from the second measurement depth zone R2 is selected by changing the gain of the preamplifier 42 at the gain changing circuit 46.

In consequence, reception signals associated with the first measurement depth zone R1 are subjected to A/D conversion in the first A/D conversion executable range in the first ultrasonic beam transmission and reception process, while reception signals associated with the second measurement depth zone R2 are subjected to A/D conversion in the second A/D conversion executable range in the second ultrasonic beam transmission and reception process.

The first and second A/D conversion executable ranges are so specified as to overlap each other.

In FIG. 4, the first and second A/D conversion executable ranges are depicted on an ultrasonic echo intensity basis.

Operations of the apparatus of Embodiment 1 are detailed below.

When the measurement region to be subjected to ultrasonography is inputted by an operator from the operating unit 29 of the diagnostic apparatus body 2, the apparatus body controller 28 divides the measurement region into a first measurement depth zone R1 at a shallower location and a second measurement depth zone R2 at a deeper location, and controls by wireless communication the individual reception signal processors 4 of the ultrasound probe 1 through the reception controller 9 so that the first A/D conversion executable range corresponding to the intensity range of ultrasonic echos returning from the first measurement depth zone R1 may be selected. The first A/D conversion executable range may be set by adjusting, in each reception signal processor 4, the gain of the preamplifier 42 at the gain changing circuit 46.

In order to determine the intensity range of ultrasonic echos from the first measurement depth zone R1 and that of ultrasonic echos from the second measurement depth zone R2, pre-scanning with an ultrasonic beam may be performed on a subject prior to diagnostic procedures to actually receive ultrasonic echos.

Upon the start of ultrasonography, the ultrasound transducers 3 transmit a first ultrasonic beam in accordance with driving signals fed from the transmission driver 7 of the ultrasound probe 1, and the reception signals as outputted from the ultrasound transducers 3 having received ultrasonic echos from the first measurement depth zone R1 in the subject are fed to the corresponding reception signal processors 4, respectively.

In each reception signal processor 4, the reception signal is amplified at the preamplifier 42, and inputted to the A/D converter 44 after high frequency components are removed from the signal at the low pass filter 43. With the first A/D conversion executable range having been selected by the adjustment of the gain of the preamplifier 42 by the gain changing circuit 46, a reception signal associated with an ultrasonic echo from the first measurement depth zone R1 is A/D converted with a resolution depending on the dynamic range of the A/D converter 44.

Sample data is generated by such A/D conversion of a reception signal at the A/D converter 44 in each reception signal processor 4, and the sample data is made serial at the parallel/serial converter 5 before being transmitted from the wireless communication unit 6 to the diagnostic apparatus body 2 in a wireless manner. The sample data as received at the wireless communication unit 21 of the diagnostic apparatus body 2 is converted at the serial/parallel converter 22 into parallel data, then stored in the data storage unit 23.

Subsequently, in each reception signal processor 4, the gain of the preamplifier 42 is increased at the gain changing circuit 46 by the reception controller 9 of the ultrasound probe 1 based on the instruction from the apparatus body controller 28 of the diagnostic apparatus body 2, so as to select the second A/D conversion executable range corresponding to the intensity range of ultrasonic echos returning from the second measurement depth zone R2 at a deeper location.

The ultrasound transducers 3 transmit a second ultrasonic beam identical to the first one on the same sound ray in accordance with driving signals fed from the transmission driver 7 of the ultrasound probe 1, and the reception signals as outputted from the ultrasound transducers 3 having received ultrasonic echos from the second measurement depth zone R2 this time are fed to the corresponding reception signal processors 4, respectively.

In each reception signal processor 4, the reception signal is amplified at the preamplifier 42, and inputted to the A/D converter 44 after high frequency components are removed from the signal at the low pass filter 43. With the second A/D conversion executable range having been selected by the change of the gain of the preamplifier 42 by the gain changing circuit 46, a reception signal associated with an ultrasonic echo from the second measurement depth zone R2 is A/D converted with a resolution depending on the dynamic range of the A/D converter 44.

The sample data as generated by the A/D conversion of a reception signal at the A/D converter 44 in each reception signal processor 4 is made serial at the parallel/serial converter 5, then transmitted from the wireless communication unit 6 to the diagnostic apparatus body 2 in a wireless manner, converted at the serial/parallel converter 22 of the diagnostic apparatus body 2 into parallel data, and stored in the data storage unit 23.

In this way, sample data associated with the first measurement depth zone R1 and that associated with the second measurement depth zone R2 on each sound ray are sequentially stored in the data storage unit 23. When sample data for one frame have been stored in the data storage unit 23, the stored sample data corresponding to the first and second measurement depth zones R1 and R2 are used in the image producer 24 to generate an image signal, and an ultrasound diagnostic image is displayed on the monitor 26 by the display controller 25 based on the image signal.

As described above, the measurement region is divided into the first and second measurement depth zones R1 and R2, and reception signals associated with the first measurement depth zone R1 are subjected to A/D conversion in the first A/D conversion executable range in the first ultrasonic beam transmission and reception process, while reception signals associated with the second measurement depth zone R2 are subjected to A/D conversion in the second A/D conversion executable range in the second ultrasonic beam transmission and reception process. Consequently, the dynamic range of the A/D converter 44 in each reception signal processor 4 can fully be utilized, which allows the generation of an ultrasound image of high quality and the achievement of power savings.

In Embodiment 1, an ultrasonic beam is transmitted twice on one and the same sound ray corresponding to the first and second measurement depth zones R1 and R2. An ultrasound image may be produced with no reduction in frame rate by, for instance, making each of the first and second ultrasonic beams so wide as to have a narrow portion spanning two sound ray areas to thereby carry out ultrasound transmission and reception for every two sound rays.

### Embodiment 2

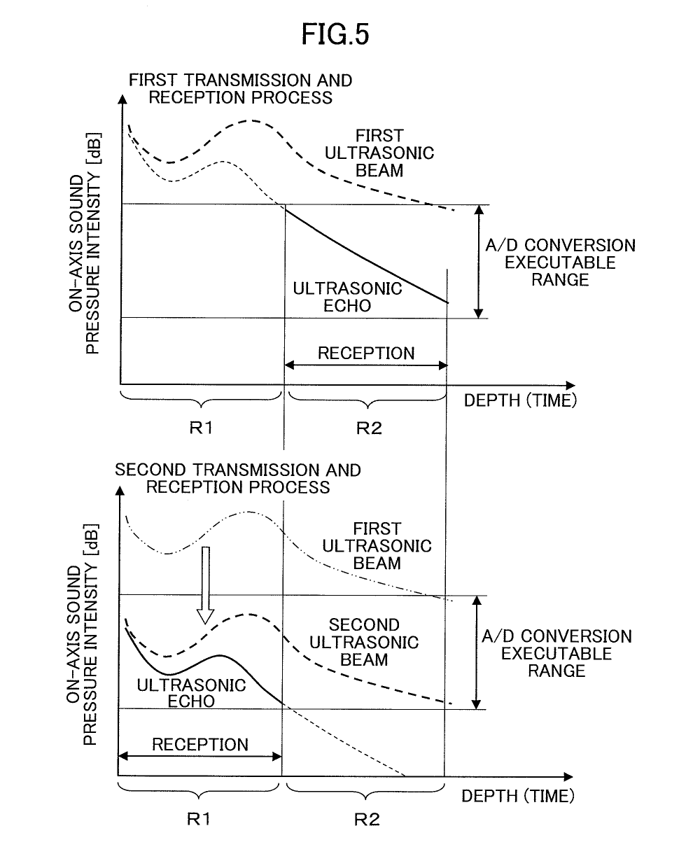

In Embodiment 1 as described above, A/D conversion of reception signals associated with ultrasonic echos from the entire depth of the measurement region is carried out by transmitting the identical ultrasonic beam in the first and second ultrasonic beam transmission and reception processes, and displacing the first A/D conversion executable range relating to the first measurement depth zone R1 and the second A/D conversion executable range relating to the second measurement depth zone R2 from each other. In Embodiment 2, as shown in FIG. 5, the intensity of the ultrasonic beam to be transmitted is changed between first and second ultrasonic beam transmission and reception processes by changing the amplitude of the ultrasonic beam, while applying the same A/D conversion executable range by each reception signal processor 4 in the two processes, so as to carry out the A/D conversion of reception signals associated with ultrasonic echos from the entire depth of a measurement region.

Similar to Embodiment 1, the apparatus body controller 28 divides the measurement region into a first measurement depth zone R1 at a shallower location and a second measurement depth zone R2 at a deeper location. In a first ultrasonic beam transmission and reception process transmitting a first ultrasonic beam, reception signals associated with the second measurement depth zone R2 at a deeper location are subjected to A/D conversion in the A/D conversion executable range as specified in each reception signal processor 4, and sample data associated with the second measurement depth zone R2 is stored in the data storage unit 23 of the diagnostic apparatus body 2.

Then, a second ultrasonic beam having an amplitude smaller than that of the first ultrasonic beam is transmitted from the ultrasound transducers 3 in accordance with driving signals fed from the transmission driver 7 under the control of the transmission controller 8 of the ultrasound probe 1 based on the instruction from the apparatus body controller 28 of the diagnostic apparatus body 2. As a result, ultrasonic echos lower in intensity than those obtained in the first transmission and reception process transmitting the first ultrasonic beam are obtained. Reception signals outputted from the ultrasound transducers 3 having received ultrasonic echos from the first measurement depth zone R1 at a shallower location are fed to the corresponding reception signal processors 4, respectively, and A/D converted with a resolution depending on the dynamic range of the A/D converter 44, and sample data associated with the first measurement depth zone R1 is stored in the data storage unit 23 of the diagnostic apparatus body 2.

The sample data thus stored in the data storage unit 23 that are associated with the first and second measurement depth zones R1 and R2 are used in the image producer 24 to generate an image signal, and an ultrasound diagnostic image is displayed on the monitor 26 by the display controller 25 based on the image signal.

In Embodiment 2, the dynamic range of the A/D converter 44 in each reception signal processor 4 can fully be utilized, which allows the generation of an ultrasound image of high quality and the achievement of power savings, as is the case with Embodiment 1.

The intensity of the ultrasonic beam to be transmitted from the ultrasound transducers 3 in accordance with driving signals fed from the transmission driver 7 may also be changed by changing one or both of the wave number and the center frequency of the ultrasonic beam instead of or along with its amplitude.

### Embodiment 3

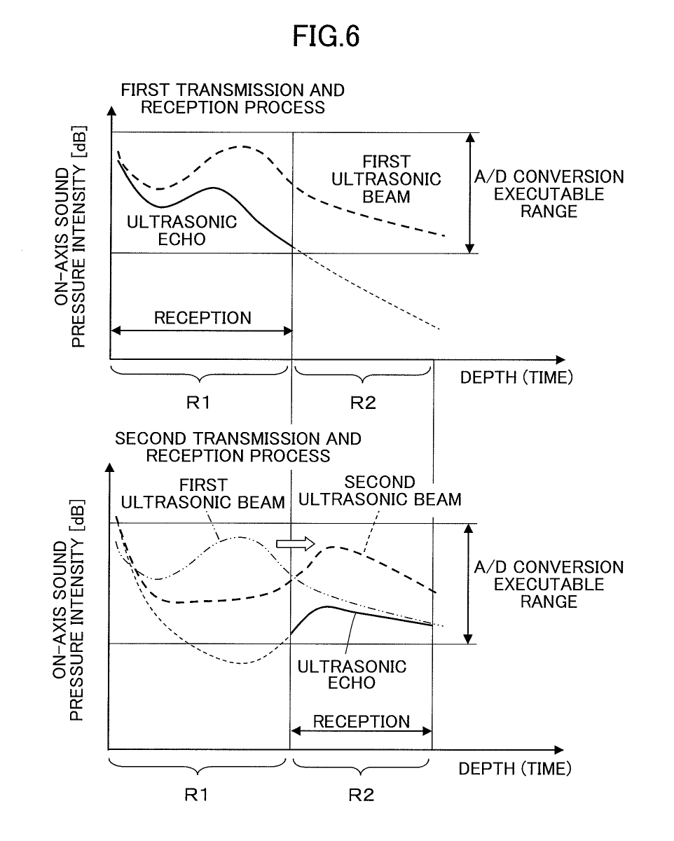

In Embodiment 2 as described above, A/D conversion of reception signals associated with ultrasonic echos from the entire depth of the measurement region is carried out by changing the amplitude of the ultrasonic beam to be transmitted between the first and second ultrasonic beam transmission and reception processes while applying the same A/D conversion executable range by each reception signal processor 4 in the two processes. It is also possible to change not the amplitude but the focal position of the ultrasonic beam to be transmitted as shown in FIG. 6.

Similar to Embodiments 1 and 2, the apparatus body controller 28 divides a measurement region into first and second measurement depth zones R1 and R2. In a first ultrasonic beam transmission and reception process transmitting a first ultrasonic beam, reception signals associated with the first measurement depth zone R1 are subjected to A/D conversion in the A/D conversion executable range as specified in each reception signal processor 4, and sample data associated with the first measurement depth zone R1 is stored in the data storage unit 23 of the diagnostic apparatus body 2.

Then, a second ultrasonic beam is transmitted from the ultrasound transducers 3 in accordance with driving signals fed from the transmission driver 7 under the control of the transmission controller 8 of the ultrasound probe 1 based on the instruction from the apparatus body controller 28 of the diagnostic apparatus body 2 so that it may differ from the first ultrasonic beam in focal position. As a result, ultrasonic echos different in waveform from those derived from the first ultrasonic beam are obtained. Reception signals outputted from the ultrasound transducers 3 having received ultrasonic echos from the second measurement depth zone R2 are fed to the corresponding reception signal processors 4, respectively, and A/D converted with a resolution depending on the dynamic range of the A/D converter 44, and sample data associated with the second measurement depth zone R2 is stored in the data storage unit 23 of the diagnostic apparatus body 2.

The sample data thus stored in the data storage unit 23 that are associated with the first and second measurement depth zones R1 and R2 are used in the image producer 24 to generate an image signal, and an ultrasound diagnostic image is displayed on the monitor 26 by the display controller 25 based on the image signal.

It should be noted that the focal position of the second ultrasonic beam needs to be formed so that ultrasonic echos from the second measurement depth zone R2 may have intensities corresponding to the A/D conversion executable range by the reception signal processor 4.

In Embodiment 3, the dynamic range of the A/D converter 44 in each reception signal processor 4 can fully be utilized, which allows the generation of an ultrasound image of high quality and the achievement of power savings, as is the case with Embodiments 1 and 2.

The intensity of the ultrasonic beam to be transmitted is controlled differently between the first and second transmissions by changing the amplitude of the ultrasonic beam in Embodiment 2, or by changing the focal position of the ultrasonic beam in Embodiment 3, although the present invention is not limited thereto. The intensity of the ultrasonic beam to be transmitted may also be controlled by changing the number of the transducers of an array transducer which are used for the transmission of an ultrasonic beam, namely, the aperture width of the array transducer. For this reason, similar effects will be achieved if the transmission driver 7 is controlled so that the ultrasound transducers 3 which are used for the transmission of an ultrasonic beam may vary in number with measurement depth zones. As a typical example, the transmission driver 7 is controlled so that fewer ultrasound transducers 3 may be used to transmit an ultrasonic beam corresponding to a measurement depth zone at a shallower location.

In addition, it is possible to change simultaneously two or more out of the amplitude of the ultrasonic beam to be transmitted, the focal position of the ultrasonic beam, and the number of the ultrasound transducers 3 to be used between the first and second transmissions.

While the measurement region is divided into two, the first and second measurement depth zones R1 and R2, in Embodiments 1 through 3 as described above, a measurement region may be divided into three or more measurement depth zones each allowing reception signals within an amplitude range narrower than the A/D conversion executable range by the reception signal processor 4. In that case, transmission of an ultrasonic beam is performed three or more times corresponding to the individual measurement depth zones so as to achieve A/D conversion of all the reception signals associated with ultrasonic echos from the entire depth of the measurement region.

Also in that case, if a plurality of ultrasonic beams sequentially transmitted on the same sound ray corresponding to the measurement depth zones are each made so wide as to have a narrow portion spanning two or more sound ray areas to thereby carry out ultrasound transmission and reception for every two or more sound rays, an ultrasound image is generated with no reduction in frame rate.

In Embodiments 2 and 3 as above, the gain changing circuit 46 as shown in FIG. 2 is unnecessary because the same A/D conversion executable range by each reception signal processor 4 is used in the first and second ultrasonic beam transmission and reception processes, that is to say, the A/D conversion executable range is not changed between the two processes.

In Embodiments 1 through 3 as above, the ultrasound probe 1 and the diagnostic apparatus body 2 are connected with each other through wireless communication, although the present invention is not limited thereto. It is also possible to connect the ultrasound probe 1 with the diagnostic apparatus body 2 via a connection cable. In that case, the wireless communication unit 6 and the communication controller 10 of the ultrasound probe 1, the wireless communication unit 21 and the communication controller 27 of the diagnostic apparatus body 2, and so forth are unnecessary.

What is claimed is:

1. An ultrasound diagnostic apparatus comprising:
an array transducer;
a transmission driver for transmitting an ultrasonic beam from the array transducer toward a subject;
reception signal processors for acquiring reception data by processing reception signals outputted from the array transducer having received an ultrasonic echo from the subject, the reception signal processors having an A/D conversion executable range narrower than an amplitude range of reception signals obtained from an entire depth of a measurement region;
an image producer for producing an ultrasound image based on the reception data acquired in the receptiOn signal processors; and
a controller for dividing the measurement region into a plurality of measurement depth zones each allowing reception signals within an amplitude range narrower than the A/D conversion executable range of the reception signal processors, controlling the transmission driver so that transmission of an ultrasonic beam from the array transducer may be performed plural times corresponding to the measurement depth zones, and controlling the reception signal processors so that reception data associated with each measurement depth zone may be acquired based on the ultrasonic beam as transmitted corresponding to the relevant measurement depth zone.

2. The ultrasound diagnostic apparatus according to claim 1, wherein the controller controls the reception signal processors so that amplification of reception signals may vary in gain with the measurement depth zones.

3. The ultrasound diagnostic apparatus according to claim 2, wherein the controller controls the reception signal processors so that reception signals allowed by a measurement depth zone at a deeper location may be amplified with a higher gain.

4. The ultrasound diagnostic apparatus according to claim 1, wherein the controller controls the transmission driver so that the ultrasonic beam transmitted corresponding to the measurement depth zones may vary in amplitude.

5. The ultrasound diagnostic apparatus according to claim 4, wherein the controller controls the transmission driver so that an ultrasonic beam with a smaller amplitude may be transmitted corresponding to a measurement depth zone at a shallower location.

6. The ultrasound diagnostic apparatus according to claim 1, wherein the controller controls the transmission driver so that the ultrasonic beam transmitted corresponding to the measurement depth zones may vary in focal position.

7. The ultrasound diagnostic apparatus according to claim 1, wherein the controller controls the transmission driver so that transducers of the array transducer which are used for the transmission of an ultrasonic beam may vary in number with the measurement depth zones.

8. The ultrasound diagnostic apparatus according to claim 7, wherein the controller controls the transmission driver so that fewer transducers of the array transducer may be used to transmit an ultrasonic beam corresponding to a measurement depth zone at a shallower location.

9. An ultrasound image producing method comprising the steps of:
transmitting an ultrasonic beam from an array transducer toward a subject based on a driving signal fed from a transmission driver;
acquiring reception data by processing reception signals outputted from the array transducer in response to ultrasonic echos from an entire depth of a measurement region in reception signal processors having an A/D conversion executable range narrower than an amplitude range of the reception signals;
producing an ultrasound image based on the acquired reception data;
dividing the measurement region into a plurality of measurement depth zones each allowing reception signals within an amplitude range narrower than the A/D conversion executable range of the reception signal processors;
controlling the transmission driver so that transmission of an ultrasonic beam from the array transducer may be performed plural times corresponding to the measurement depth zones; and
controlling the reception signal processors so that reception data associated with each measurement depth zone may be acquired based on the ultrasonic beam as transmitted corresponding to the relevant measurement depth zone.
